(19) United States
(12) Patent Application Publication
(10) Pub. No.: US 2013/0133646 A1
(43) Pub. Date: May 30, 2013

(54) HELMET-TYPE OF PATIENT INTERFACE DEVICE AND METHOD USE

(71) Applicants: Catherine Michelle Rose, Methuen, MA (US); Matthew Joseph Schnaderbeck, Methuen, MA (US)

(72) Inventors: Catherine Michelle Rose, Methuen, MA (US); Matthew Joseph Schnaderbeck, Methuen, MA (US)

(73) Assignee: KONINKLIJKE PHILIPS ELECTRONICS N.V., EINDHOVEN (NL)

(21) Appl. No.: 13/581,867

(22) PCT Filed: Feb. 24, 2011
(86) PCT No.: PCT/IB11/50779

§ 371 Date: Feb. 12, 2013

Related U.S. Application Data
(60) Provisional application No. 61/309,607, filed on Mar. 2, 2010.

(51) Int. Cl. A61M 16/06; A61M 16/00

(52) U.S. Cl. CPC A61M 16/0683; A61M 16/0066
USPC 128/201.23

(57) ABSTRACT

A positive airway pressure support system (2, 7, 130) that includes a pressure generating device (4) structured to produce an intermittent or continuous flow of breathing gas at a selected positive pressure or pressures, and a patient interface device (2, 72, 132) operatively coupled to the pressure generating system and structured to deliver the flow of breathing gas to an airway of a patient. The patient interface device includes a helmet (30) structured to be worn on the head of the patient and a patient interface element (22) coupleable to the top portion (44), the left side portion (82) and/or the right side portion (84) of the helmet in manner which stabilizes the patient interface element relative to the helmet and resists forces applied to the patient interface element when the patient interface device is donned by the patient and the patient moves while in a supine position.

This patent application claims the priority benefit under 35 U.S.C. §119(e) of U.S. Provisional Application No. 61/309,607 filed on Mar. 2, 2010, the contents of which are herein incorporated by reference.

The present invention relates to positive airway pressure support systems, and, in particular, to patient interface devices for communicating a flow of to an airway of a user in which the patient interface device includes a helmet-style headgear adapted to support a mask on a user and to an associated method of using such a patient interface system.

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle, e.g., bi-level and flex therapies, and/or varies based on the monitored condition of the user, e.g., auto-titration therapies. Such therapies are typically provided to the patient at night while the patient is sleeping.

Non-invasive ventilation and pressure support therapies, as just described, involve the placement of a patient interface device including a mask component on the face of a patient. The mask component may be, without limitation, a nasal mask that covers the patient's nose, a nasal cannula having nasal prongs that are received within the patient's nares, a nasal/oral (full face) mask that covers the nose and mouth, or a total face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Treating certain patients, such as young children and the elderly, that suffer from OSA or other respiratory disorders can be difficult as those patients are often unable and/or unwilling to fall asleep while wearing the patient interface device required for treatment. For example, young children may be uncooperative because they do not understand that, while the patient interface device may be slightly uncomfortable, it is necessary for proper treatment. As a result, young children suffering from OSA are traditionally treated using a tracheostomy, wherein an incision is made into the trachea, through the neck, and a tube is inserted so as to make an artificial opening for the passage of air into the airway of the patient. Tracheostomies, while effective, are difficult to maintain because they require frequent suctioning (24 hours a day) and because the tracheostomy site, if not properly cared for, can become infected, bleed or develop inflammatory issues. In addition, people with tracheostomies often have difficulty with speech and swallowing. Interfering with swallowing may impact nutrition and require additional surgery to provide a g-tube for patient feeding.

In addition, with current patient interface devices employing known headgear configurations, there is chance that the mask will be caused to move and a seal between the mask and the patient's face will be broken as a result of forces against the mask generated by patient movement while the patient is sleeping. These forces are often counteracted by applying stronger strapping forces with the headgear. However, in current patient interface devices, such increased strapping forces are borne largely be the head/face of the patient, which can result in discomfort and/or facial/head deformation/flattening.

In one embodiment, a positive airway pressure support system for treating a respiratory disorder is provided that includes a pressure generating device having a controller, wherein the pressure generating device is structured to produce an intermittent or continuous flow of breathing gas at a selected positive pressure or pressures under control of the controller, and a patient interface device operatively coupled to the pressure generating system and structured to deliver the flow of breathing gas to an airway of a patient. The patient interface device includes a helmet structured to be worn on the head of the patient, the helmet having a forehead portion structured to cover at least a portion of the forehead of the patient, a top portion structured to cover at least a portion of the top of the head of the patient, a rear portion structured to cover at least a portion of the rear of a head of the patient, and left and right side portions each structured to cover at least a portion of a respective side of the head of the patient, and a patient interface element selectively coupleable to at least one of the top portion, the left side portion and the right side portion of the helmet in manner which stabilizes the patient interface element relative to the helmet and resists forces applied to the patient interface element when the patient interface device is donned by the patient and the patient moves while in a supine position.

In another embodiment, a method of treating a respiratory disorder is provided that includes placing a helmet on the head of a patient while the patient is awake, the helmet having a forehead portion structured to cover at least a portion of the forehead of the patient, a top portion structured to cover at least a portion of the top of the head of the patient, a rear portion structured to cover at least a portion of the rear of a head of the patient, and left and right side portions each structured to cover at least a portion of a respective side of the head of the patient, waiting for the patient to fall asleep while wearing the helmet, while the patient is sleeping, attaching a patient interface element to the helmet, wherein the patient interface element is attached in a position wherein a cushion or other patient sealing member is in contact with the face of the patient, and after the patient interface element has been attached to the helmet, providing intermittent or continuous positive pressure to the airway of the patient via the patient interface element.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

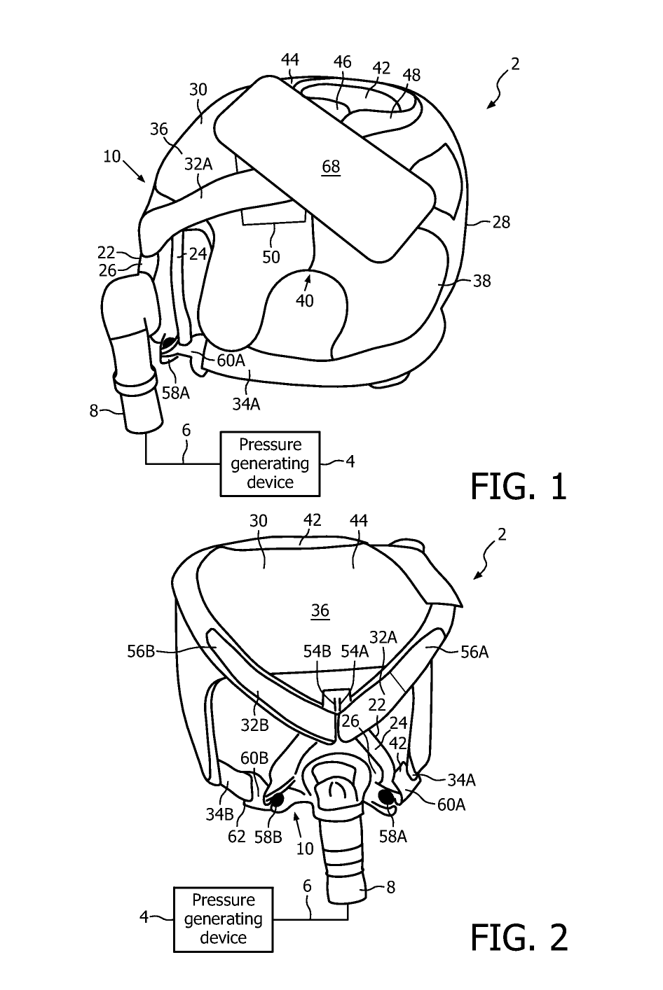

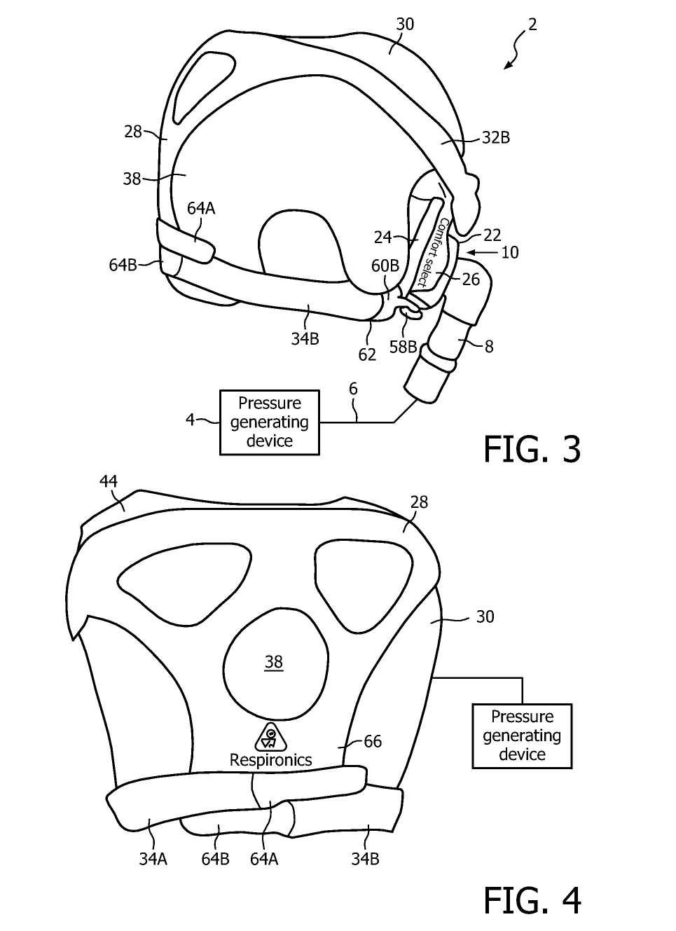

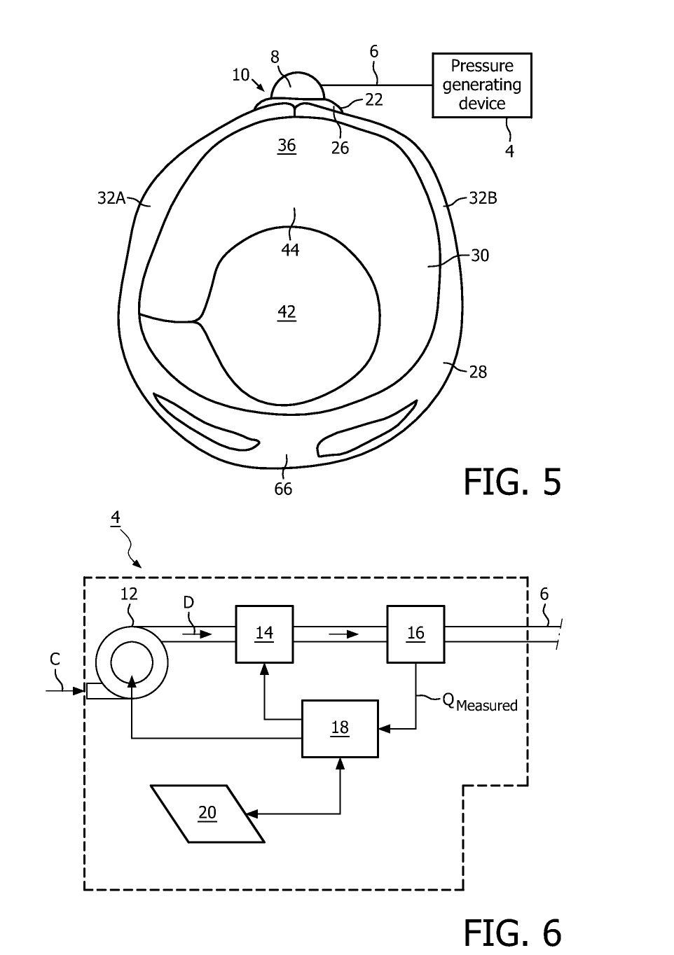

FIGS. 1-5 are side elevational, front elevational, rear isometric, rear elevational and top plan views of a pressure support system adapted to provide a regimen of respiratory therapy to a patient according to one particular, non-limiting embodiment of the invention;

FIG. 6 is a schematic diagram of one embodiment of a pressure generating device that may be employed in the pressure support system of FIGS. 1-5;

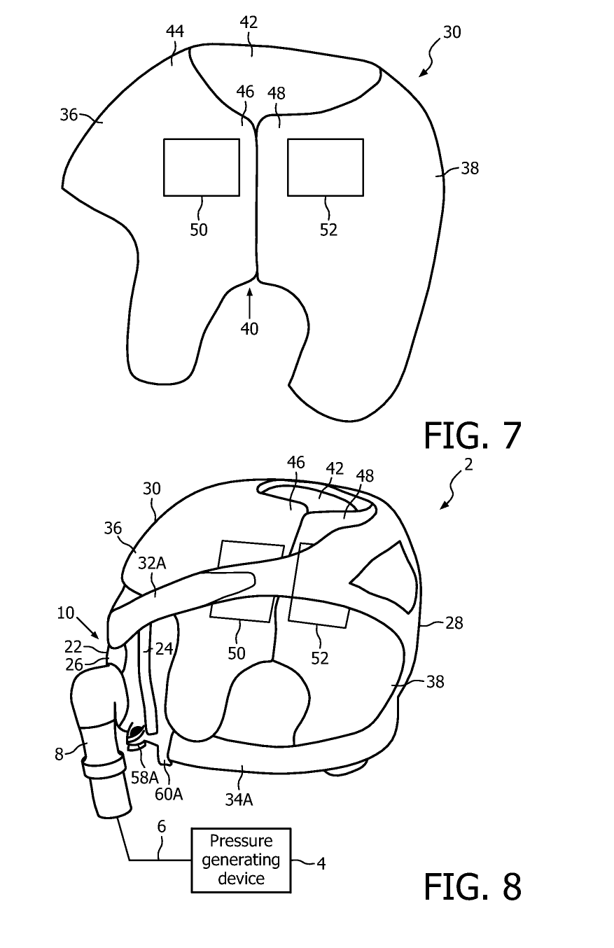

FIG. 7 is a left side elevational view of a helmet that may be used in the pressure support system of FIGS. 1-5;

FIG. 8 is a left side elevational view of the pressure support system of FIGS. 1-5 illustrating how the headgear and mask thereof may be coupled to the helmet thereof;

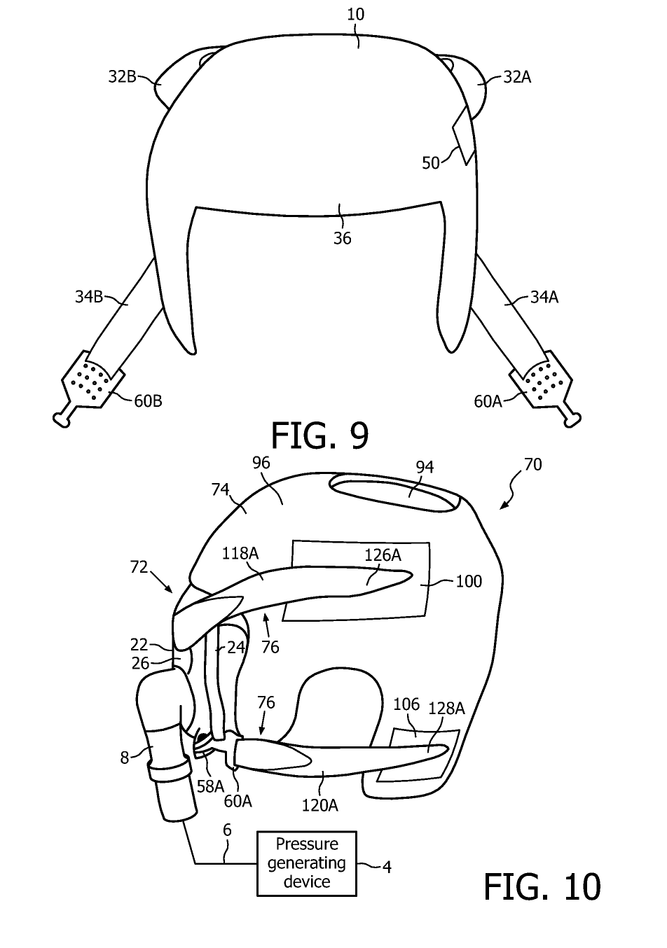

FIG. 9 is a front elevational view of the patient interface device of the pressure support system of FIGS. 1-5 in a condition in which it may be worn while a user thereof falls asleep;

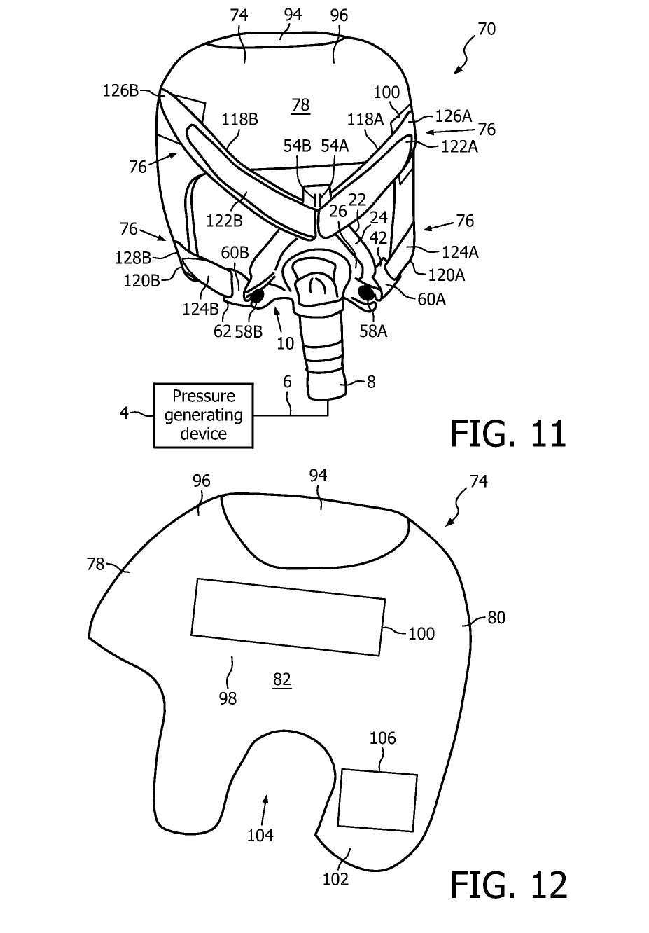

FIGS. 10-11 are side elevational and front views, respectively, of a pressure support system adapted to provide a regimen of respiratory therapy to a patient according to a second particular, non-limiting embodiment of the invention;

FIGS. 12-14 are left side elevational, right side elevational and rear elevational views, respectively, of a helmet forming part of the patient interface device of FIG. 10;

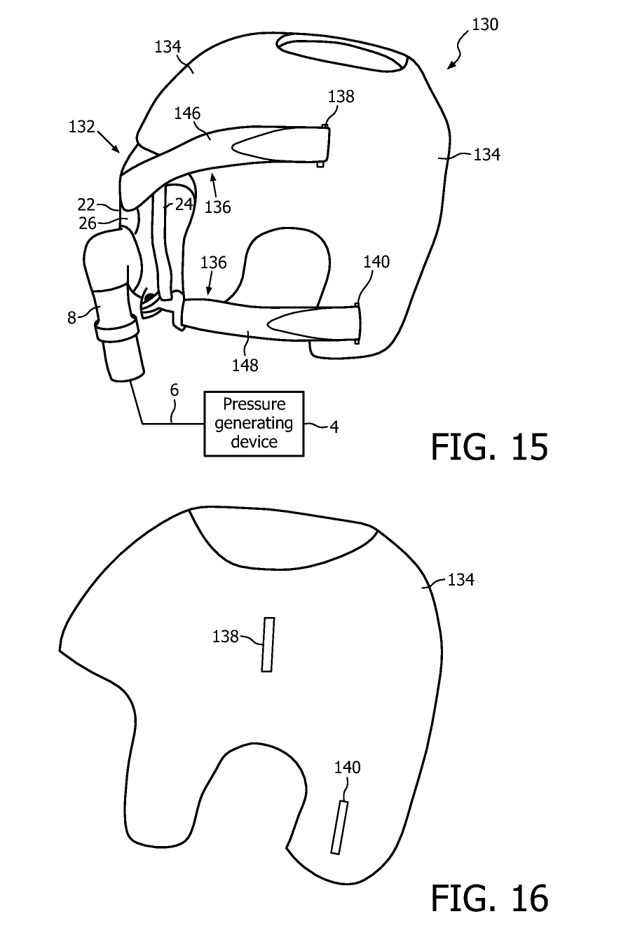

FIG. 15 is a side elevational view and FIG. 16 is a front view of a pressure support system adapted to provide a regimen of respiratory therapy to a patient according to a third particular, non-limiting embodiment of the invention;

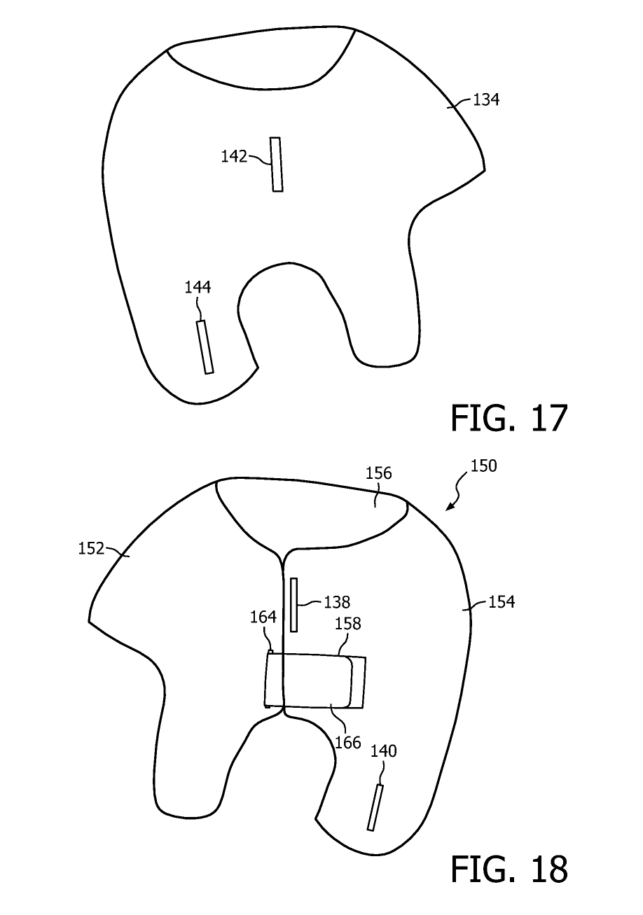

FIGS. 16-17 are left side elevational and right side elevational views, respectively, of a helmet that may be used in the pressure support system of FIGS. 15 and 16; and

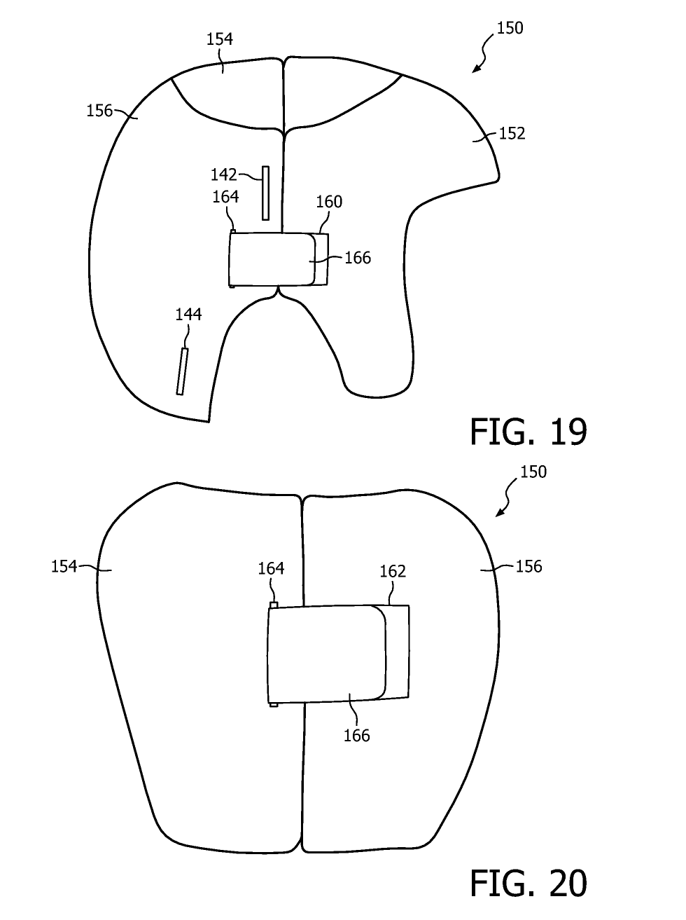

FIGS. 18-20 are left side elevational, right side elevational and rear elevational views, respectively, of a helmet according to an alternative embodiment that may be used in the pressure support system of FIGS. 15 and 16.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIGS. 1-5 are side elevational, front elevational, rear isometric, rear elevational and top plan views of pressure support system 2 adapted to provide a regimen of respiratory therapy to a patient according to one particular, non-limiting embodiment of the invention. Pressure support system 2 includes pressure a generating device 4, a delivery conduit 6 coupled to an elbow connector 8, and a patient interface device 10. Pressure generating device 4 is structured to generate an intermittent or continuous flow of positive pressure breathing gas and may include, without limitation, ventilators, constant pressure support devices (such as a continuous positive airway pressure device, or CPAP device), variable pressure support devices (e.g., BiPAp®, Bi-Flex®, or C-Flex™ devices manufactured and distributed by Philips Respironics of Murrysville, Pa.), and auto-titration pressure support devices. Patient circuit 6 is structured to communicate the flow of breathing gas from pressure generating device 4 to patient interface device 10 through conduit 6 and elbow connector 8.

FIG. 6 is a schematic diagram of one, non-limiting embodiment of pressure generating device 4 that may be employed in pressure support system 2. Referring to FIG. 6, pressure generating device 4 includes a gas flow generator 12, such as a blower used in a conventional CPAP or bi-level pressure support device, which receives breathing gas, generally indicated by arrow C, from any suitable source, e.g., a pressurized tank of oxygen or air, the ambient atmosphere, or a combination thereof. Gas flow generator 12 generates a flow of breathing gas, such as air, oxygen, or a mixture thereof, for delivery to an airway of a patient at relatively higher and lower pressures, i.e., generally equal to or above ambient atmospheric pressure. The pressurized flow of breathing gas, generally indicated by arrow D from gas flow generator 12, is delivered via delivery conduit 6 and elbow connector 8 to patient interface device 10 and ultimately to the airway of the patient. Delivery conduit 6, elbow connector 8 and patient interface device 10 are typically collectively referred to as a patient circuit.

In the illustrated embodiment, pressure generating device 4 includes a pressure controller in the form of a valve 14 provided in delivery conduit 6. Valve 14 controls the pressure of the flow of breathing gas from flow generator 12 that is delivered to the patient. For present purposes, flow generator 12 and valve 14 are collectively referred to a pressure generating system because they act in concert to control the pressure and/or flow of gas delivered to the patient. However, it should be apparent that other techniques for controlling the pressure of the gas delivered to the patient, such as varying the blower speed of flow generator 12, either alone or in combination with a pressure control valve, are contemplated by the present invention. Thus, valve 14 is optional depending on the technique used to control the pressure of the flow of breathing gas delivered to the patient. If valve 14 is eliminated, the pressure generating system corresponds to flow generator 12 alone, and the pressure of gas in the patient circuit is controlled, for example, by controlling the motor speed of flow generator 12.

In the illustrated embodiment, pressure generating device 4 further includes a flow sensor 16 that measures the flow of the breathing gas within delivery conduit 6. In the particular embodiment shown in FIG. 6, flow sensor 16 is interposed in line with delivery conduit 6, for example downstream of valve 14. Flow sensor 16 generates a flow signal Q_{MEASURED} that is provided to a controller 18 and is used by controller 18 to determine the flow of gas delivered to the patient. Of course, other techniques for measuring the respiratory flow of the patient are contemplated by the present invention, such as, without limitation, measuring the flow directly at the patient or at other locations along delivery conduit 6, measuring patient flow based on the operation of flow generator 12, and measuring patient flow using a flow sensor upstream of valve 14.

Controller 18 may be, for example, a microprocessor, a microcontroller or some other suitable processing device, that includes or is operatively coupled to a memory (not shown) that provides a storage medium for data and software executable by controller 18 for controlling the operation of pressure generating device 4. Finally, input/output device 20 is provided for setting various parameters used by pressure generating device 4, such as the desired pressure settings, as well as for displaying and outputting information and data to a user, such as a clinician or caregiver.

In the exemplary embodiment, pressure support system 2 functions as either a CPAP system or a bi-level pressure support system, and, therefore, includes all of the capabilities necessary in such systems in order to provide an intermittent or continuous positive pressure to the patient or separate IPAP and EPAP levels to the patient. In the case of a bi-level pressure support system, U.S. Pat. No. 5,148,802 to Sanders et al., U.S. Pat. No. 5,313,937 to Zdrojkowski et al., U.S. Pat. No. 5,433,193 to Sanders et al., U.S. Pat. No. 5,632,269 to Zdrojkowski et al., U.S. Pat. No. 5,803,065 to Zdrojkowski et al., U.S. Pat. No. 6,029,664 to Zdrojkowski et al., and U.S. Pat. No. 6,920,875 to Hill et al., the contents of each of which are incorporated herein by reference, describe how to accomplish the necessary functions in order to provide separate IPAP and EPAP levels to the patient. These functions include techniques for detecting and estimating leak, and techniques for detecting the respiratory state of a patient, and managing, e.g., triggering and cycling, the bi-level delivery of breathing gas to the patient in the presence of leaks. Thus, a detailed discussion of these functions is omitted from the present application for the sake of simplicity and brevity

Returning to FIGS. 1-5, patient interface device 10 includes a patient interface element 22, which, in the exemplary embodiment, is a nasal mask. However, any type of patient interface element, such as a nasal/oral mask, a nasal cannula, or a full face mask, which facilitates the delivery of the flow of breathing gas to the airway of a patient, may be used as patient interface element 22 while remaining within the scope of the present invention. In this illustrated exemplary embodiment, patient interface element 22 is a conventional mask that includes a cushion 24 coupled to a stiff shell 26. An opening in shell 26, to which elbow connector 8 is coupled, allows the flow of breathing gas from pressure generating device 4 to be communicated to an interior space defined by shell 26 and cushion 4, and then to the airway of a patient.

Patient interface device 10 further includes a headgear component 28 that is securely attached to a helmet-type of element 30. In the illustrated embodiment, headgear component 28 includes first and second top straps 32A, 32B and first and second bottom straps 34A, 34B. Other configurations are also possible, including, without limitation, configurations including two straps, three straps or five or more straps, and configurations including one or more lever arms coupled to patient interface element 22, with to without one or more straps associated straps.

FIG. 7 is a side elevational view of helmet 30 according to one particular, non-limiting embodiment. As used herein, the term “helmet” shall mean a component structured to be worn on the head that includes at least a forehead portion structured to cover at least a portion of the forehead of the wearer, a top portion structured to cover at least a portion of the top of the head of the wearer, a rear portion structured to cover at least a portion of the rear of the head of the wearer, and left and right side portions each structured to cover at least a portion of a respective side of the head of the wearer.

In an exemplary embodiment of the present invention, helmet 30 is made of a stiff material such as, without limitation, a plastic material, a stiff and/or lightweight foam material or a metal covered by a foam material, and may be similar to a head-shaping helmet that is currently used for correction of plagiocephaly (flattening of one side of the occiput) or a foam helmet used by seizure and/or balance disorder patients. As seen in FIG. 7, helmet 30 includes a front portion 36 and rear portion 38. A separation 40 is provided between front portion 36 and rear portion 38 on one side of helmet 30 to enable the size of helmet 30 to be adjusted by allowing terminal ends 46, 48 of front portion 36 and rear portion 38, respectively, to move relative to one another. In addition, in the illustrated embodiment, a hole 42 is provided in top portion 44 of helmet 30. It should be appreciated that helmet configurations without such a hole and/or without such a separation may also be used.

The present invention contemplates that the helmets can be made from common components that are connected in an adjustable manner, e.g., all forehead portions have the same size and shape and adjustably coupled to the other portions of the helmet. The present invention also contemplates that one or more of the components of the helmet can have different sizes or shapes. For example, the may be two or different “styles” of forehead portions. The user or caregiver selection the style (size and shape) forehead portion best suited to their forehead configuration and uses that selected forehead portion with the other portions of helmet. This selected forehead pad can be adjustably coupled to the other portions of the headgear. Finally, the present invention still further contemplates that one or more (or all) of the portions of the headgear can be customized to fit a particular user. For example, the single-piece type of helmet shown in FIG. 10 can be custom-formed to fit a particular user.

As described in greater detail elsewhere herein, the exemplary embodiment of patient interface device 10 employs a hook and loop fastening system, such as VELCRO®, to secure headgear component 28 and patient interface element 22 to helmet 30. In particular, terminal end 46 has hook connector patch 50 adhered thereto and terminal end 48 has hook connector patch 52 adhered thereto. As described in greater detail herein, hook connector patches 50, 52 are structured to be selectively connected to a loop connector portion that is provided on the underside of the first top strap 32A of headgear component 28.

As best seen in FIG. 2, shell 26 of patient interface element 22 includes strap loops 54A and 54B for enabling top straps 32A, 32B to be connected to patient interface element 22. In particular, the exterior of each of top straps 32A and 32B includes a loop fastener portion, and a corresponding hook fastener portion is provided on the exterior of each of end portions 56A, 56B of top straps 32A and 32B. Thus, top strap 32A may be threaded through strap loop 54A and then bent back on itself to adhere the hook fastener portion of end portion 56A of top strap 32A to the loop fastener portion provided on the exterior of top strap 32A. Similarly, top strap 32B may be threaded through strap loop 54B and then bent back on itself to adhere the hook fastener portion of end portion 56B of top strap 32B to the loop fastener portion provided on the exterior of top strap 32B. The hook and loop fastening system just described allows the length of top straps 32A, 32B to be adjusted, and thus the size of headgear component 28 to be partially adjusted. Of course, the present invention contemplates the fastening system may use other forms of connection other than hook and loop fasteners such as snaps or buckles.

In addition, shell 26 portion of patient interface element 22 includes slots 58A, 58B, each structured to receive and releasably hold a catch of a respective clip element 60A, 60B attached to bottoms straps 34A, 34B. In the exemplary embodiment, slots 58A, 58B and clip elements 60A, 60B are structured in the form of a ball and socket configuration, although it will be appreciated that other configurations for attaching the straps to the mask are also possible and contemplated by the present invention. Each clip element 60A, 60B also includes loop 62 for receiving a respective bottoms strap 34A, 34B of headgear component. In addition, bottoms straps 34A, 34B each include a respective terminal end 64A, 64B provided with a hook fastener component for connecting to a loop fastener portion (not shown) provided on back portion 66 of headgear component 28. This hook and loop fastening system allows the length of bottoms straps 34A, 34B to be adjusted, and thus the size of headgear component 28 to be partially adjusted.

In order to attach patient interface device 10 to helmet 30, the size of headgear component 28 is first adjusted so that it fits snugly around the exterior of helmet 30 by adjusting top straps 32A, 32b and bottom straps 34A, 34B as just described. When so adjusted, patient interface device 10 will able to be fit over and around helmet 30 as shown in FIG. 8, which, as described elsewhere herein, is the configuration and orientation that patient interface device 10 and helmet 30 will be in when respiratory therapy is provided to the patient by pressure generating device 4. When patient interface device 10 is fitted to helmet 30 in this manner, the loop connector portion that is provided on the underside of the first top strap 32A of headgear component 28 is attached to hook connector patches 50, 52 to securely hold headgear component 28 and patient interface device 10 attached thereto in place on helmet 30.

Although hook connector patches 50, 52 are shown in the illustrated embodiment as being provided only on one side of helmet 30, it will be appreciated that similar patches may be provide don the other side of helmet 30 for attaching to a loop connector portion that is provided on the underside of the second top strap 32b in order to provide increased attachment force. In addition, in the illustrated embodiment, optional strip 68 having a loop connector portion provided on the underside thereof may be placed over top strap 32A as shown in FIG. 1 and connected to hook connector patches 50, 52 to provide still additional attachment force.

As described elsewhere herein, some individuals, such as children, are unable and/or unwilling to fall asleep while wearing a patient interface device for treatment of a sleeping disorder such as sleep apnea (for example, children may be uncooperative because they do not understand that, while the patient interface device may be slightly uncomfortable, it is necessary for proper treatment). According to one exemplary embodiment, pressure support system 2 described herein may be used in a method that facilitates and encourages use by such individuals during sleep. In particular, according to this method, helmet 30 is placed on the head of the individual in a condition wherein patient interface element 22 is positioned away from the face of the individual and elbow connector is detached from conduit 6.

For example, and without limitation, helmet 30 may be placed on the head of the individual in a condition wherein bottom straps 34A, 34B are detached from shell 26 of patient interface element 22 and allowed to hang to the side and wherein patient interface element 22 is positioned on/adjacent to the top rear portion of helmet 30 out of the way of the individual's face (patient interface element 22 will still move as the wearer moves). This condition is illustrated in FIG. 9. Alternatively, helmet 30 may be placed on the head of the individual in a condition wherein top strap 32A and bottom strap 34A are detached from shell 26 of patient interface element 22, which allows patient interface element 22 to hang to one side of helmet 30 and away from the individual's face.

The individual is then allowed to fall asleep naturally with patient interface element 22 positioned away from their face. Once the individual falls asleep, a caregiver, such as a parent, nurse or personal care attendant (PCA), may then reposition patient interface element 22 such that patient interface element 22 is properly positioned to provide therapy (e.g., covering the individual's nose in the case of a nasal mask) and connect elbow connector 8 to conduit 6. If the configuration illustrated in FIG. 9 is employed, this process will involve moving patient interface element 22 down into position and attaching bottom straps 34A, 34B to shell 26 as described elsewhere herein. If the alternative resting condition of patient interface device 10 described above is employed, this process will involve moving patient interface element 22 over into position and attaching top strap 42A and bottom strap 34A to shell 26 as described elsewhere herein. In addition, in the repositioning process, care is taken to ensure that the loop connectors of top strap 32A of headgear component 28 are attached to hook connector patches 50, 52 to securely hold headgear component 28 and patient interface device 10 attached thereto in place on helmet 30. The caregiver then activates pressure generating device 4 to begin the provision of therapy. As will appreciated, this method allows the individual to fall asleep without the discomfort caused by patient interface element 22 contacting their face and without the noise generated by pressure generating device 4, with therapy beginning only after the individual has fallen asleep.

Furthermore, patient interface device 10 is additionally beneficial due to the fact that, when worn, helmet 30 is fixed in position relative to the wearer's head, and patient interface element 22 is fixed in position relative to helmet 30 (in the embodiment of FIGS. 1-5, patient interface element 22 has four points of fixation to helmet 30). This results in the immobilization of patient interface element 22 relative to the patient and helmet 30 in response to forces that may be applied to patient interface element 22 during sleep (e.g., the wearer, while in a supine position, may move around in bed and forces may be applied to patient interface element 22 as a result). Such immobilization resists breaking of the seal between patient interface element 22 and the wearer's face, which is important to the provision of effective therapy.

In addition, strapping forces and pressures applied by top straps 32A, 32B and bottom straps 34A, 34B are largely borne by helmet 30 and not the head (including the face) of the wearer since contact by top straps 32A, 32B and bottom straps 34A, 34B with the head will be minimal or nonexistent. This helps to reduce the possibility of facial/head deformation/flattening and/or facial obtrusions (red marks, welts, blisters) that may otherwise result from such forces (this is particularly important in the case of young children that may not have fully developed bone structures). The strapping forces may be adjusted by adjusting top straps 32A, 32B and bottom straps 34A, 34B. The total strapping force is a combination of the lifting force due to the intermittent or continuous positive pressure and the force required to create and maintain a seal. With normal, known fabric headgear, the majority of the force is applied to the back of the wearer's head. The large contact area of helmet 30 (and the other helmet embodiments described herein) increases the distribution of the forces which reduces the likelihood of any skull/face deformation and reduces the strapping force needed for stability. For example, on a large full-face mask at a positive pressure of 20 cmH₂0, each strap would only pull with approximately 1 lb force if the present invention is employed.

FIGS. 10-11 are side elevational and front views, respectively, of a pressure support system 70 adapted to provide a regimen of respiratory therapy to a patient according to a second particular, non-limiting embodiment of the invention. Pressure support system 70 includes many of the components that form part of pressure support system 2, and like components are labeled with like reference numerals. Pressure support system 70 includes a patient interface device 72 that includes a patient interface element 22 having a cushion 24 and a shell 26, helmet 74, and headgear component 76 that is securely and removeably attached to both patient interface element 22 and helmet 74.

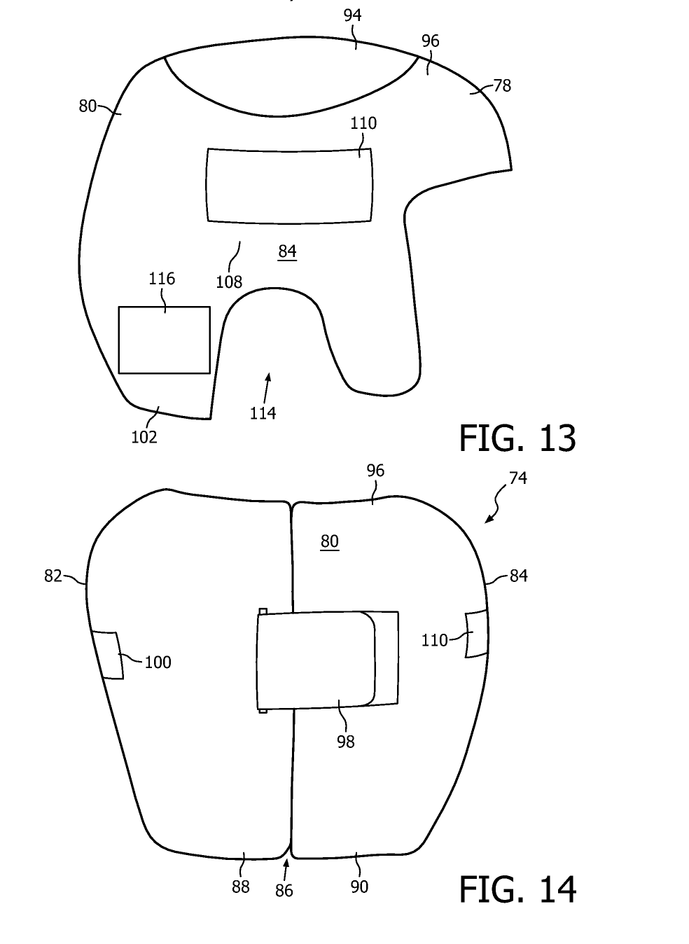

FIGS. 12-14 are left side elevational, right side elevational and rear elevational views, respectively, of helmet 74. Helmet 74 includes a front portion 78, a rear portion 80, a left side portion 82, and a right side portion 84. Referring to FIG. 14, separation 86 is provided in rear portion 80 to enable the size of helmet 74 to be adjusted by allowing terminal ends 88, 90 of rear portion 80 to move relative to one another. A fastening system 92, which in the illustrated embodiment is a hook an loop fastening system, is provided on rear portion 80 to prevent further movement of terminal ends 88, 90 when the desired size is achieved. In addition, a hole 94 is provided in top portion 96 of helmet 74. Referring to FIG. 12, central portion 98 of left side portion 82 has hook connector patch 100 adhered thereto, and lower rear portion 102 (located behind cutout portion 104 for receiving a left ear) of left side portion 82 has hook connector patch 106 adhered thereto. Similarly, referring to FIG. 13, central portion 108 of right side portion 84 has hook connector patch 110 adhered thereto, and lower rear portion 112 (located behind cutout portion 114 for receiving a right ear) of left side portion 84 has hook connector patch 116 adhered thereto.

Referring to FIGS. 10 and 11, headgear component 76 includes first and second top straps 118A, 118B and first and second bottom straps 120A, 120B. The exterior of each of top straps 118A and 118B and bottom straps 120A and 120B includes a loop fastener portion, and a corresponding hook fastener portion is provided on the exterior of each of end portions 122A, 122B of top straps 118A and 118B and end portions 124A, 124B of bottom straps 120A and 120B. Thus, top strap 118A may be threaded through strap loop 54A and then bent back on itself to adhere the hook fastener portion of end portion 122A of top strap 118A to the loop fastener portion provided on the exterior of top strap 118A. Similarly, top strap 118B may be threaded through strap loop 54B and then bent back on itself to adhere the hook fastener portion of end portion 122B of top strap 118B to the loop fastener portion provided on the exterior of top strap 118B. Bottom straps 120A, 120B may be coupled to respective clip element 60A, 60B in a similar manner. The hook and loop fastening systems just described allows the length of top straps 118A, 118B and the length of bottom straps 120A, 120B to be adjusted.

In addition, rear end 126A, 126B of top straps 118A, 118B and rear end rear end 128A, 128B of bottom straps 120A, 120B are each provided with a loop fastener portion on the underside thereof. Patient interface element 22 may thus be attached to helmet 74 by attaching the loop fastener portion of rear end 126A of top strap 118A to hook connector patch 100, the loop fastener portion of rear end 126B of top strap 118B to hook connector patch 110, the loop fastener portion of rear end 128A of bottom strap 120A to hook connector patch 106, and the loop fastener portion of rear end 128B of bottom strap 120B to hook connector patch 116 at appropriate locations to provide the desired fit. Fit may also be adjusted by adjusting the length of top straps 118A, 118B and bottom straps 120A, 120B.

Like pressure support system 2, pressure support system 70 may be used in a method that facilitates and encourages use by such individuals during sleep. In particular, helmet 74 is placed on the head of the individual in a condition wherein either top strap 118A and bottom strap 120A or top strap 118B and bottom strap 120B are detached from shell 26 of patient interface element 22, which allows patient interface element 22 to hang to one side of helmet 74 and away from the individual's face. The individual is then allowed to fall asleep naturally with patient interface element 22 positioned away from their face. Once the individual falls asleep, a caregiver, such as a parent, then repositions patient interface element 22 such that patient interface element 22 is properly positioned to provide therapy by attaching the detached straps as described elsewhere herein. The caregiver also connects elbow connector 8 to conduit 6 and then activates pressure generating device 4 to begin the provision of therapy. This method allows the individual to fall asleep without the discomfort caused by patient interface element 22 contacting their face and without the noise generated by pressure generating device 4, with therapy beginning only after the individual has fallen asleep. In addition, patient interface device 72 as just described provides the same fixation and immobilization benefits that were discussed elsewhere herein in connection with patient interface device 10.

FIG. 15 is a side elevational view of a pressure support system 130 adapted to provide a regimen of respiratory therapy to a patient according to a third particular, non-limiting embodiment of the invention. Pressure support system 130 includes many of the components that form part of pressure support systems 2 and 70, and like components are labeled with like reference numerals. Pressure support system 130 includes a patient interface device 132 that includes a patient interface element 22 having a cushion 24 and shell 26, a helmet 134, and a headgear component 136 that is securely and removeably attached to both patient interface element 22 and helmet 134.

FIGS. 16-17 are left side elevational and right side elevational views, respectively, of helmet 134. Helmet 134 is similar to helmet 74, except that instead of hook connector patches 100, 106, 110, 116, helmet 134 includes loops 138, 140, 142, 144 for enabling headgear component 136 to securely attach patient interface element 22 to helmet 134. In particular, headgear component 136 includes top straps 146, one on each side of patient interface element 22, and bottom straps 148, one on each side of patient interface element 22, that at one end are connected to shell 26 of patient interface element 22. The opposite end of each of top straps 146 and bottom straps 148 is provided with a hook and loop fastening system that enables each of top straps 146 and bottom straps 148 to be inserted through a respective loop 138, 140, 142, 144 and folded back on itself as described elsewhere herein to secure patient interface element 22 to helmet 134 with a desired level of force. The use of pressure support system 130 in a method that facilitates and encourages use by such individuals during sleep is substantially similar to that described above in connection with pressure support system 70.

Alternative connection mechanisms for coupling a plurality of straps, such as top straps 146 and bottoms straps 148, to a helmet, such as helmet 134, are also contemplated, including, without limitation, snaps or buckles.

FIGS. 18-20 are left side elevational, right side elevational and rear elevational views, respectively, of helmet 150 according to an alternative embodiment that may be used in pressure support system 130. Helmet 150 is a three-part helmet and includes front portion 152, left rear portion 154, and right rear portion 156. Front portion 152, left rear portion 154, and right rear portion 156 are adjustably connected to one another by first, second and third hook and loop fastening systems 158, 160, 162 (each of which includes a loop 164 and a connecting strap 166 as shown).

It can be appreciated that the present invention provides a solution to the need for an effective, non-invasive system and method for treating patients, such as young children, adults and the elderly, suffering from OSA or a similar respiratory disorder that are unable and/or unwilling to fall asleep while wearing the patient interface device that is required for treatment. Moreover, the present invention addresses the need for a patient interface device that provides effective mask stability (against forces that may be generated by patient movement during sleep) without applying a significant strapping force to the head/face of the patient.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A positive airway pressure support system for treating a respiratory disorder, comprising:
(a) a pressure generating device having a controller, wherein the pressure generating device is structured to produce an intermittent or continuous flow of breathing gas at a selected positive pressure or pressures under control of the controller; and
(b) a patient interface device operatively coupled to the pressure generating system and structured to deliver the flow of breathing gas to an airway of a patient, the patient interface device comprising:
(1) a helmet structured to be worn on a head of the patient, the helmet being made of a stiff material selected from a group consisting of (i) a plastic material, (ii) a foam material, and (iii) a metal material at least partially covered by a foam material, the helmet having a forehead portion structured to cover at least a portion of a forehead of the patient, a top portion structured to cover at least a portion of a top of the head of the patient, a rear portion structured to cover at least a portion of a rear of a head of the patient, and left and right side portions each structured to cover at least a portion of a respective side of the head of the patient;
(2) a patient interface element selectively coupleable to at least one of the top portion, the left side portion and the right side portion of the helmet in manner that stabilizes the patient interface element relative to the helmet and resists forces applied to the patient interface element when the patient interface device is donned by the patient and the patient moves while in a supine position.

2. The positive airway pressure support system according to claim 1, wherein the patient interface element is selectively coupleable to at least the left and right side portions of the helmet.

3. The positive airway pressure support system according to claim 2, wherein the forehead portion and the left and right side portions define a facial opening structured to accommodate a face of the patient when the helmet is donned by the patient, wherein the patient interface element is selectively movable from a first position wherein the patient interface element is connected to the helmet and removed from the facial opening to a second position, and wherein the patient interface element is secured to the left and right side portions of the helmet and covers at least a portion of the facial opening.

4. The positive airway pressure support system according to claim 2, wherein the patient interface device includes a first strap, a second strap, a third strap and a fourth strap, wherein the first and second straps are connected to a first side of the patient interface element and are selectively connectable to the left side portion of the helmet, and wherein the third and fourth straps are connected to a second side of the patient interface element opposite the first side of the patient interface element and are selectively connectable to the right side portion of the helmet.

5. The positive airway pressure support system according to claim 4, wherein the first and second straps are selectively connectable to the left side portion of the helmet by a first hook and loop fastener system provided on the first strap and the left side of the helmet and a second hook and loop fastener system provided on the second strap and the left side of the helmet, and wherein the third and fourth straps are selectively connectable to the right side portion of the helmet by a third hook and loop fastener system provided on the third strap and the right side of the helmet and a fourth hook and loop fastener system provided on the fourth strap and the right side of the helmet.

6. The positive airway pressure support system according to claim 4, wherein the first strap is selectively connectable to a first loop provided on the left side portion of the helmet, the second strap is selectively connectable to a second loop provided on the left side portion of the helmet, the third strap is selectively connectable to a third loop provided on the right side portion of the helmet, and the fourth strap is selectively connectable to a fourth loop provided on the right side portion of the helmet.

7. The positive airway pressure support system according to claim 4, wherein a length of each of the first strap, the second strap, the third strap and the fourth strap is selectively adjustable.

8. The positive airway pressure support system according to claim 4, wherein the second strap is releasably connected to the first side of the patient interface element at a bottom of the first side of the patient interface element and the fourth strap is releasably connected to the second side of the patient interface element at a bottom of the second side of the patient interface element.

9. The positive airway pressure support system according to claim 2, wherein the patient interface device includes a cushion and a headgear component including a first strap, a second strap, a third strap and a fourth strap, wherein the first and second straps are connected to a first side of the patient interface element and the third and fourth straps are connected to a second side of the patient interface element opposite the first side of the patient interface element, wherein the headgear component is selectively connectable to the helmet, and wherein responsive to the patient interface device being donned by the patient in a manner wherein the first and second straps are coupled to the left side of the helmet and the third and fourth straps are coupled to right side of the helmet and the cushion is in sealing contact with a face of the patient, the helmet and each of the straps of the headgear component are structured to ensure that strapping forces provided by each of the straps are largely borne by the helmet and that each of the straps will provide no more than 1 pound of strapping force to a head of the patient.

10. The positive airway pressure support system according to claim 9, wherein the second strap is releasably connected to the first side of the patient interface element at a bottom of the first side of the patient interface element and the fourth strap is releasably connected to the second side of the patient interface element at a bottom of the second side of the patient interface element.

11. The positive airway pressure support system according to claim 9, wherein responsive to the patient interface device being donned by the patient in a manner wherein the first and second straps are coupled to the left side of the helmet and the third and fourth straps are coupled to right side of the helmet and the cushion is in sealing contact with a face of the patient, the helmet and each of the straps of the headgear component are structured to ensure that contact with the head of the patient by each of the straps is nonexistent.

12. The positive airway pressure support system according to claim 1, wherein a size of the helmet is selectively adjustable.

13. A method of treating a respiratory disorder, comprising:
placing a helmet on a head of a patient while the patient is awake, the helmet being made of a stiff material selected from a group consisting of a plastic material, a foam material, and a metal material covered by a foam material, the helmet having a forehead portion structured to cover at least a portion of a forehead of the patient, a top portion structured to cover at least a portion of a top of the head of the patient, a rear portion structured to cover at least a portion of a rear of a head of the patient, and left and right side portions each structured to cover at least a portion of a respective side of the head of the patient;
waiting for the patient to fall asleep while wearing the helmet;
while the patient is sleeping, attaching a patient interface element to the helmet such that a patient sealing portion of the patient interface element is in contact with the face of the patient; and
after the patient interface element has been attached to the helmet, providing a flow of gas to the airway of the patient through via the patient interface element.

14. The method according to claim 13, wherein the attaching comprises attaching the patient interface element to at least the left and right sides of the helmet.

15. The method according to claim 13, wherein the forehead portion and the left and right side portions define a facial opening structured to accommodate a face of the patient when the helmet is placed in the head of the patient, wherein the attaching comprises moving the patient interface element from a first position wherein the patient interface element is connected to the helmet and removed from the facial opening to a second position wherein the patient interface element is secured to the left and right side portions of the helmet and covers at least a portion of the facial opening.

16. The method according to claim 15, wherein in the first position, the patient interface element rests at least partially on one or both of the top portion of the helmet and the rear portion of the helmet.

17. The method according to claim 15, wherein in the first position, the patient interface element is connected to only one of the left side portion and the right side portion of the helmet.

18. The method according to claim 13, wherein the mask is attached to the helmet by a headgear component having a plurality of straps, and wherein following the attaching step the helmet and each of the straps of the headgear component are structured to ensure that strapping forces provided by each of the straps are largely borne by the helmet and that each of the straps will provide no more than 1 pound of strapping force to a head of the patient.

19. The method according to claim 18, wherein following the attaching step the helmet and each of the straps of the headgear component are structured to ensure that contact with the head of the patient by each of the straps is nonexistent.
